(19) United States
(12) Patent Application Publication
(10) Pub. No.: US 2021/0244462 A1
(43) Pub. Date: Aug. 12, 2021

(54) ELECTROSURGICAL SYSTEM, ELECTROSURGICAL INSTRUMENT, METHOD OF WRITING OPERATIONAL DATA, AND ELECTROSURGICAL SUPPLY DEVICE

(71) Applicant: OLYMPUS WINTER & IBE GMBH, Hamburg (DE)

(72) Inventors: Lutz KERSTEN, Berlin (DE); Anne KWIK, Berlin (DE)

(73) Assignee: OLYMPUS WINTER & IBE GMBH, Hamburg (DE)

(21) Appl. No.: 17/172,733

(22) Filed: Feb. 10, 2021

(30) Foreign Application Priority Data
Feb. 10, 2020 (DE) 10 2020 103 278.1

(51) Int. Cl. A61B 18/14; A61B 18/12; G06F 3/06; G16H 20/40

(52) U.S. Cl. CPC A61B 18/14; A61B 18/1206; G06F 3/0655; A61B 18/16; G06F 3/0673; G16H 20/40; G06F 3/0604

(57) ABSTRACT

An electrosurgical system includes: an electrosurgical supply device, and an electrosurgical instrument, wherein the electrosurgical instrument includes a memory element that can be read out and written to by the electrosurgical supply device when the electrosurgical instrument is connected to the electrosurgical supply device, and the electrosurgical supply device is configured to write operational data to the memory element. The electrosurgical system is characterized in that the operational data is or can be stored on the memory element in a data structure which includes a first operational data element and a second operational data element, and in that the electrosurgical supply device is configured to write operational data alternately to the first operational data element or the second operational data element.

The invention relates to an electrosurgical system comprising: an electrosurgical supply device, and an electrosurgical instrument, wherein the electrosurgical instrument comprises a memory element that can be read out and written to by the electrosurgical supply device when the electrosurgical instrument is connected to the electrosurgical supply device, and the electrosurgical supply device is configured to write operational data to the memory element. The invention further relates to an electrosurgical instrument of a corresponding electrosurgical system, a method for writing operational data, and an electrosurgical supply device.

Electrosurgical systems are being used in surgery for some time to perform various procedures. In electrosurgery in the narrow sense, tissue to be treated is directly exposed to electric currents, which are usually high-frequency alternating currents. By appropriate dimensioning of the instruments and the currents and voltages used, different tissue effects may be achieved, for example coagulation or cutting of the tissue.

Systems are also known in which a tissue to be treated is additionally or exclusively subjected to ultrasound via an ultrasound sonotrode. In this case, there is usually a transducer in the instrument to be used, which converts an electrical signal provided by a supply device in the form of an ultrasound generator into ultrasonic oscillations of the sonotrode. Such ultrasound systems are also considered to be electrosurgical systems in terms of the invention.

Electrosurgical instruments generally have a limited service life. This is equally true for instruments that are specified for single use only, as well as for instruments that are intended for multiple uses. Various methods have been developed to ensure that an electrosurgical instrument is not used beyond its intended number of uses.

In modern electrosurgical systems, the instrument is equipped with a memory element on which configuration data for the supply device is stored. As a component of the configuration data, an operational data element is included in which the electrosurgical supply device may write operational data. This operational data may be consumption data, such as number and duration of activations, delivered energy/power, timestamps, temperature data, or the like. Once an electrosurgical instrument is connected to an electrosurgical supply device, the supply device may read out the operational data element and determine whether further use of the instrument is permissible.

Particularly in the field of electrosurgery, electromagnetic interference may cause a writing access of the electrosurgical supply device to the memory element in the electrosurgical instrument to fail. In this case, the operational data stored on the electrosurgical instrument may be compromised, so it can no longer be reliably determined whether further use of the electrosurgical instrument is permissible.

It is therefore an object of the invention to provide an electrosurgical system which is improved with respect to the problems described.

This object is achieved according to the invention by an electrosurgical system comprising: an electrosurgical supply device, and an electrosurgical instrument, wherein the electrosurgical instrument comprises a memory element that can be read out and written to by the electrosurgical supply device when the electrosurgical instrument is connected to the electrosurgical supply device, and the electrosurgical supply device is configured to write operational data to the memory element, wherein the operational data is or can be stored on the memory element in a data structure which comprises a first operational data element and a second operational data element, and wherein the electrosurgical supply device is configured to write operational data alternately to the first operational data element or the second operational data element.

By the described development, even in the event of a failed write access to one of the operational data elements, the other operational data element remains accessible, so that the operational data previously stored there may still be read. Thus, if necessary, the state of use of the electrosurgical instrument may be determined accurately enough to decide on further use.

In a possible further development of the invention, the data structure may comprise a write flag the content of which indicates whether operational data is to be written to the first operational data element or the second operational data element.

In this regard, the electrosurgical supply device may be configured to toggle the write flag after operational data has been successfully written to the first operational data element or the second operational data element.

In a preferred development of an electrosurgical system according to the invention, in the data structure a write flag may be associated with each of the operational data elements, and the electrosurgical supply device may be configured to toggle, after successfully writing operational data to one of the operational data elements, the write flag associated with the respective operational data element.

In this regard, the electrosurgical supply device may preferably be configured to write operational data to the first operational data element when the values of the write flags are not equal, and to write operational data to the second operational data element when the values of the write flags are equal.

According to a second aspect of the invention, the object is achieved by an electrosurgical instrument of an electrosurgical system according to the above embodiments. With respect to the advantages and effects achievable thereby, reference is explicitly made to the above description.

According to a third aspect of the invention, the object is achieved by a method for writing operational data to a memory element of an electrosurgical instrument in an electrosurgical system according to the above embodiments, comprising the steps of: reading the value of a write flag from a data structure stored on the memory element; determining, based on the value of the write flag, whether operational data is to be written to a first operational data element or to a second operational data element; writing the operational data to the determined operational data element; and toggling the write flag.

The method described ensures that at any time only one of the operational data elements is accessed in write mode. In the event of a write error, there is thus a high probability that the other operational data element will remain intact, and thus the last updated operational data may be retrieved.

In a possible further development of a method according to the invention, the values of two write flags may be read, each of which is associated with one operational data element, wherein the operational data is written to the first operational data element if the values of the two write flags are not equal, and is written to the second operational data element if the values of the two write flags are equal.

Preferably, after writing operational data to one of the operational data elements, the write flag associated with the respective operational data element may be toggled.

According to a fourth aspect of the invention, the object is achieved by an electrosurgical supply device of an electrosurgical system which is configured to perform a method as described above.

The invention is described in more detail below with reference to a number of exemplary figures, wherein the embodiments shown in the figures are merely intended to assist in a better understanding of the invention without limiting it.

There are shown in:

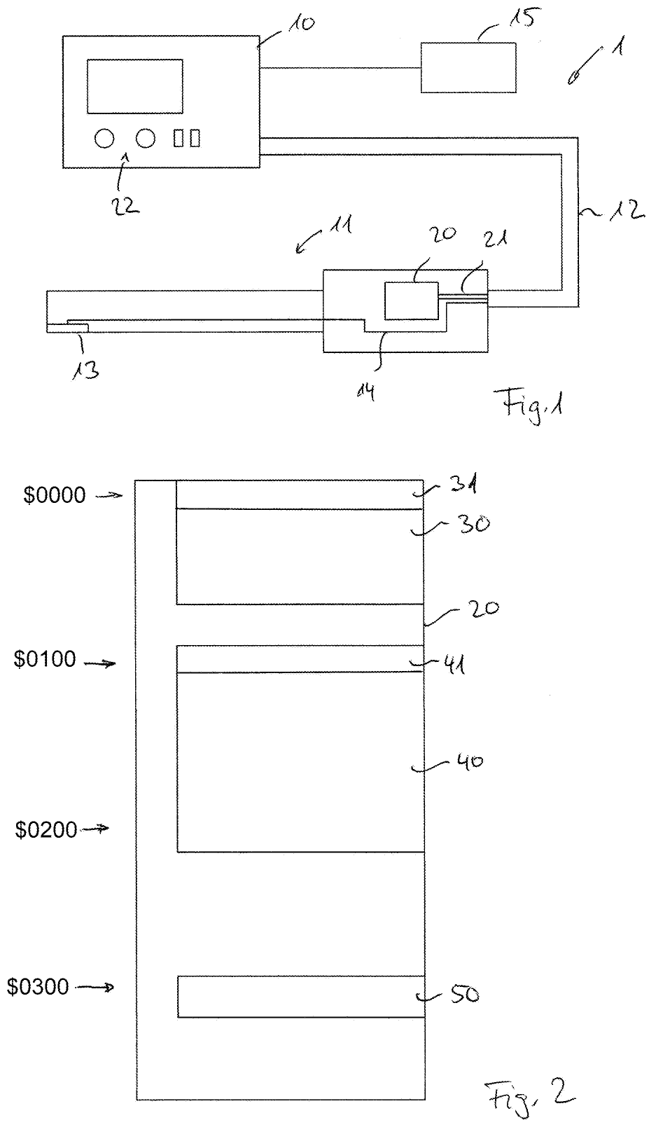

FIG. 1: an electrosurgical system,

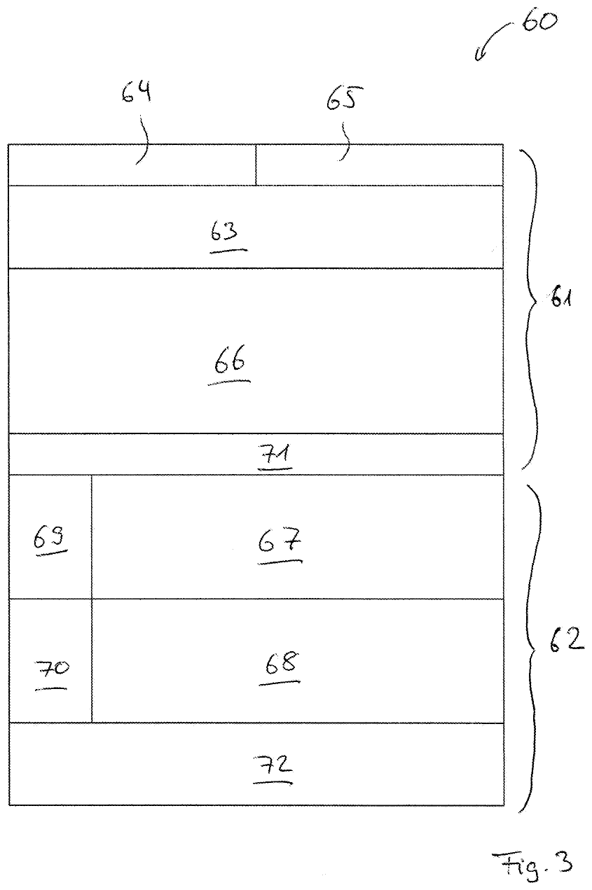

FIG. 2: a data structure,

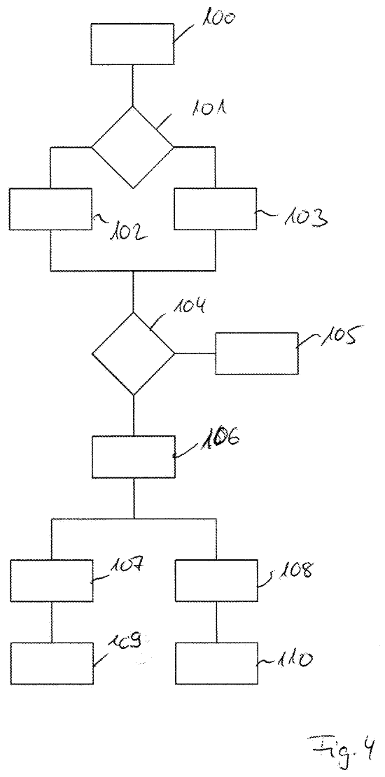

FIG. 3: a structure of a set of configuration data,

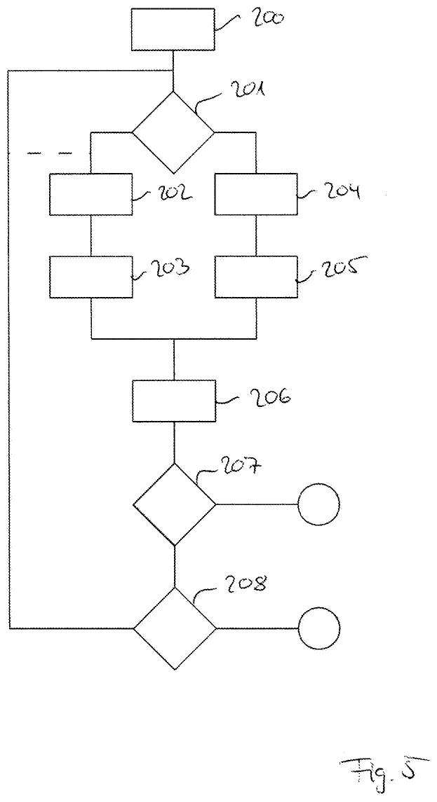

FIG. 4: a method for reading and writing operational data,

FIG. 5: a method for reading configuration data.

FIG. 1 shows an electrosurgical system 1 with an electrosurgical supply device 10 which is a high frequency generator, and with an electrosurgical instrument 11. The electrosurgical instrument 11 is connected to the electrosurgical supply device 10 via a cable 12. Instead of the depicted cable 12, the connection between the electrosurgical instrument 11 and the electrosurgical supply device 10 can also be made contactless, for example using NFC (Near Field Communication) or RFID (Radio Frequency Identification).

At a distal end of the electrosurgical instrument 11, an electrode 13 is disposed with which tissue may be treated. For this purpose, the electrode 13 is connected to the electrosurgical supply device 10 via a line 14.

The electrosurgical instrument may have more than one electrode. Alternatively or in addition to the electrode 13, the electrosurgical instrument 10 may comprise one or more ultrasound transducers.

The electrosurgical supply device 10 generates a high frequency electrical signal, which is conducted via the line 14 to the electrode 13 where it applies a therapeutic effect to tissue not shown. To complete the electrosurgical circuit, a neutral electrode 15 may be provided, which is also connected to the electrosurgical supply device 10.

The electrosurgical instrument 11 is equipped with a memory element 20 on which configuration data is stored. Once the electrosurgical instrument 11 is connected to the electrosurgical supply device 10, the electrosurgical supply device 10 reads out the memory element 20 via lines 21. The electrosurgical supply device 10 uses the configuration data read from the memory element 20 to configure the electrical signal that is delivered to the electrosurgical instrument 11. Different characteristics of the electrical signal may also be altered by control elements 22 on the electrosurgical supply device 10.

The configuration data is stored on the memory element 20 in a flexible data structure, which is shown schematically in FIG. 2. Therein, the logical content of the memory element 20 is shown with the memory address incrementing from top to bottom.

A first set 30 of configuration data is stored at a first memory address, for example at logical address $0000. A second set 40 of configuration data is stored after the first set 30, for example at logical address $0100. A termination data element 50 is placed in this case after the second set 40 of configuration data, for example at logical address $0300.

For better understanding, the logical addresses are specified in hexadecimal numbers ($ . . . ), so $0100 corresponds to a value of 256, $0200 to a value of 512, and so on.

The first set 30 of configuration data may be intended to be used with an older generation electrosurgical supply devices 10. Such supply devices expect only a single set of configuration data on memory element 20, which is always located at logical address $0000. This set of configuration data is limited in its content, as it can only contain parameters for electrosurgical instruments and waveforms that were already known when the corresponding generation of electrosurgical supply devices was developed.

For more recent instruments or waveforms, the second set 40 of configuration data is provided. A modern electrosurgical supply device 10 is capable of reading configuration data from other logical addresses of the memory element 20, and thus can access the second set 40 of configuration data.

The second set 40 of configuration data may store configuration data that supplements the configuration data of the first set 30, such that it can only be used in conjunction with that configuration data. Alternatively, the configuration data stored in the second set 40 may be complete in itself.

The logical address at which the second set 40 of configuration data is stored is dependent on the length of the first set 30 of configuration data. In this regard, the length of the first set 30 may be fixed and known, so that the logical address of the second set 40 is also known.

The first set 30 of configuration data may also be of variable length. In this case, the first set 30 includes a first length data element 31 indicating the length of the first set 30 and/or the address of the next set of configuration data.

The second set 40 of configuration data will typically always be of variable length and therefore also include a second length data element 41 indicating the length of the second set 40.

In addition to the first set 30 and the second set 40, any number of further sets of configuration data may be stored in the memory element 20. To indicate the end of the datasets, the termination data element 50 is placed after the last dataset.

The individual sets 30, 40 may be directly adjacent to each other. However, typically the memory element 20 will only be accessible for reading and/or writing in blocks, such as blocks of $0100 in length. Since the individual sets 30, 40 will not necessarily also be $0100 in length, there may be unused memory areas between individual sets 30, 40 or the termination data element 50.

FIG. 3 shows in detail a possible structure of a dataset 60 of configuration data which may be stored in the data structure on the memory element 20 instead of or in addition to the sets 30, 40.

The dataset 60 has two sections 61, 62.

The first section 61 begins with a definition data element 63.

The definition data element 63 contains information about the structure of the dataset 60, i.e. type and/or version description, length and/or position information of this and other sections within the dataset 60, and/or position information of the next set of configuration data, as well as specific information of the memory element 20, such as the block size with which it can be read from or written to the memory element. In this regard, type and/or version data may be contained in a type data element 64, and length and/or position information may be contained in a length data element 65. The type data element 64 and/or the length data element 65 may be independent data elements or sub-elements of the definition data element 63.

Using the definition data element 63, the supply device 10 is able to determine whether it is compatible with the set of data 60.

If the dataset 60 is the last set of configuration data in the data structure, $0000 may be set in the definition data element 63 as the logical memory address of the next dataset.

The definition data element 63 defines the structure of both the parameter data element 66 and the further section 62.

The parameter data element 66 contains the actual configuration data for determining the electrical signal to be delivered by the supply device 10.

The section 61 of the dataset 60 of configuration data is defined as a “read only” section, meaning that the data stored in the section 61 cannot be operationally modified by the supply device 10.

The second section 62 includes two operational data elements 67, 68. Operational data of the electrosurgical instrument 11 is stored in the operational data elements 67, 68 by the electrosurgical supply device 10. This operational data may be consumption data, for example, number and duration of activations and/or delivered energy/power by the instrument 11, time stamps, temperature data, or the like. The operational data may be evaluated by the electrosurgical supply device 10 in order to determine whether further use of the instrument 11 is permissible.

In order to allow writing to the operational data elements 67, 68 by the supply device 10, the section 62 is defined as a “read/write” section, meaning that write access by the supply device 10 is allowed.

Since errors may occur when writing to the operational data elements 67, 68, for example due to interference signals, the operational data elements 67, 68 are written to alternately by the supply device 10. In this way, it is ensured that the operational data stored in the previous write access is still available if a write access has failed.

In order to determine which of the operational data elements 67, 68 contains the most recent operational data and which is to be written to next, a write flag 69, 70 is assigned to each of the operational data elements 67, 68. After a successful write operation to one of the operational data elements 67, 68, the assigned write flag 69 or 70 is toggled, i.e. set from “zero” to “one” or from “one” to “zero”.

The supply unit 10 reads the write flags 69, 70 before each read or write access to the operational data elements 67, 68. If both write flags 69, 70 have the same value, the operational data in operational data element 67 is the most recent, and the next operational data is to be written to operational data element 68. On the other hand, if the write flags 69, 70 have different values, the operational data in the operational data element 68 is the most recent, and the next operational data is to be written to the operational data element 67.

In a factory-setting of the instrument 11, the sections 67, 68, 69 and 70 contain a predetermined sequence of numbers, for example, the Fibonacci sequence. The supply unit 10 first attempts to recognize this sequence of numbers. If these sections contain this sequence, the supply device 10 recognizes that the instrument 11 is an unused instrument.

After the first use of the instrument 11, the supply device writes operational data to the operational data element 68, and sets the write flag 70 to the value “zero”. The operational data may be usage data, for example, number and duration of activations and/or delivered energy/power by the instrument 11, timestamps, temperature data, or the like.

The next time the instrument 11 is used with the supply device 10 or with another supply device, it is now detected that the section 62 contains only the first half of the predetermined sequence of numbers, for example the Fibonacci sequence. As a result, the supply unit 10 knows that the most recent operational data is stored in the operational data element 68, and that the next operational data is to be written to the operational data element 67. The write flag 69 is set to the value “zero”.

The next time the instrument 11 is used with the supply device 10, or with another supply device, the flags 69, 70 are read. Since their values are now the same, the supply unit knows that the most recent operational data is stored in operational data element 67, and that the next operational data is to be written to operational data element 68. The write flag 70 is set to the value “one”. The next time the instrument 11 is used with the power supply unit 10, or with another supply unit, the flags 69, 70 are read again. Since their values are now not equal, the supply unit knows that the most recent operational data is stored in operational data element 68, and that the next operational data is to be written to operational data element 67.

Instead of two write flags 69, 70, a single write flag may also be used, which is toggled after each writing operation. The value of the write flag then indicates which operational data element contains the most recent operational data and which operational data element is to be written to next.

Defining individual areas of memory element 20 as “read only” or “read/write” is possible only for individual memory blocks of a predetermined size, which does not necessarily match the sizes of sections 61, 62. Therefore, unused memory areas 71, 72 may be present at the end of sections 61, 62. Similar unused memory elements, not shown, may be located between the individual data elements.

To ensure the integrity of the data stored in the single data elements, the data structure may include checksum elements, which are not shown.

A method for reading and writing operational data by a supply device 10 is shown in FIG. 4. The write flags 69, 70 are read in a first step 100 and compared with each other in a second step 101. If both write flags 69, 70 have the same value, the operational data element 67 is read out in step 102. If the values of the write flags 69, 70 are different, the operational data element 68 is read out in step 103.

In step 104, the read operational data is used to check whether further use of the instrument 11 is permissible. If this is not the case, a corresponding message is issued by the supply device 10 in step 105, and the procedure is discontinued.

If use is permissible, the instrument 11 is activated by the supply device 10 in step 106, taking into account configuration data loaded from the memory element 20 and, if applicable, any user input.

After use, based on the result of the comparison in step 101, current operational data is either written to operational data element 68 in step 107 if the values of write flags 69, 70 were the same, or current operational data is written to operational data element 67 in step 108 if the values of write flags 69, 70 were different. After successful writing, the corresponding write flag 69 (step 109) or 70 (step 110) is toggled, thus completing the procedure.

In case the data structure contains multiple sets of configuration data comprising writable operational data elements, current operational data should be written to each set individually. This is the only way to ensure that a supply device which reads only a portion of the sets of configuration data also accesses the most recent operational data.

A method for reading the sets 30, 40, 60 of operational data from the memory element 20 by the supply device 10 is shown in FIG. 5. Herein, in a first step 200, a first block of data, starting at logical address $0000, is read from the memory element. In step 201, it is checked whether the read data block contains a length data element 31 of a first set 30 of configuration data. If so, in step 202 the complete first dataset 30 is read from memory 20, and in step 203 the logical memory address of the next set of configuration data is determined based on the contents of length data element 31.

If the first block of data does not contain a length data element, in step 204 the first dataset 30 is read assuming that it has a fixed known length. Accordingly, in step 205, the logical memory address of the next set of configuration data is determined based on the known fixed length of the first set 30.

Next, in a step 206, a data block is read from the previously determined next logical memory address, and in a step 207, a check is made to determine whether this data block contains meaningful data. If the corresponding data block does not contain meaningful data, all datasets are read from the memory element 20 and the method is terminated.

In the present example, the phrase “does not contain meaningful data” may include a case where the determined logical memory address is outside the accessible memory area of the memory element 20, for example, where the first set 30 of configuration data in an older generation electrosurgical instrument almost completely fills the memory element. In this case, the software implementing the described method must be capable of intercepting any memory address error that may occur.

Moreover, the phrase “does not contain meaningful data” includes any case in which the block of read data does not include either a type data element and/or a length data element of another set of configuration data, or a data termination element.

A next step 208 then examines whether the read data block includes a data termination element 50. In this case, all sets of configuration data are also read, and the procedure is terminated.

If, on the other hand, the data block contains a type data element and/or a length data element of another set of configuration data, the method is repeated from step 201. The procedure of step 201 may be omitted from the loop if all further sets of configuration data, except for the first set 30, always contain a length data element. In this case, it may be possible to jump directly to step 202 after step 208, as indicated by the dashed line in FIG. 5.

After completion of the procedure, the supply unit 10 can determine whether the respective sets are compatible with the supply unit 10 on the basis of the contents of the type data elements that were read and only consider such compatible sets.

1. Electrosurgical system, comprising:
an electrosurgical supply device; and
an electrosurgical instrument, wherein
the electrosurgical instrument comprises a memory element that can be read out and written to by the electrosurgical supply device when the electrosurgical instrument is connected to the electrosurgical supply device, and the electrosurgical supply device is configured to write operational data to the memory element,
wherein the operational data is or can be stored on the memory element in a data structure which comprises a first operational data element and a second operational data element, and
in that the electrosurgical supply device is configured to write operational data alternately to the first operational data element or the second operational data element.

2. Electrosurgical system according to claim 1, wherein the data structure comprises a write flag, the content of which indicates whether operational data is to be written to the first operational data element or the second operational data element.

3. Electrosurgical system according to claim 2, wherein the electrosurgical supply device is configured to toggle the write flag after operational data has been successfully written to the first operational data element or the second operational data element.

4. Electrosurgical system according to claim 2, wherein in the data structure a write flag is associated with each of the operational data elements, and in that the electrosurgical supply device is configured to toggle, after successfully writing operational data to one of the operational data elements, the write flag associated with the respective operational data element.

5. Electrosurgical system according to claim 4, wherein the electrosurgical supply device is configured to write operational data to the first operational data element when the values of the write flags are not equal, and to write operational data to the second operational data element when the values of the write flags are equal.

6. Electrosurgical instrument of an electrosurgical system according to claim 1.

7. Method for writing operational data to a memory element of an electrosurgical instrument in an electrosurgical system according to claim 1, comprising the steps of:
a) reading the value of a write flag from a data structure stored on the memory element,
b) determining, based on the value of the write flag, whether operational data is to be written to a first operational data element or to a second operational data element,
c) writing the operational data to the determined operational data element, and
d) toggling the write flag.

8. Method according to claim 7, wherein the values of two write flags are read, each of which is associated with an operational data element, and wherein the operational data is written to the first operational data element if the values of the two write flags are not equal, and is written to the second operational data element if the values of the two write flags are equal.

9. Method according to claim 8, wherein, after writing operational data to one of the operational data elements, the write flag associated with the respective operational data element is toggled.

10. Electrosurgical supply device of an electrosurgical system, which is configured to perform a method according to claim 7.
